(19) United States
(12) Patent Application Publication
(10) Pub. No.: US 2007/0287836 A1
(43) Pub. Date: Dec. 13, 2007

(54) Method for Preparing Compounds Comprising Cucurbituril Groups

(76) Inventor: Anthony Ivan Day, New South Wales (AU)

Correspondence Address: STITES & HARBISON PLLC, 1199 NORTH FAIRFAX STREET, SUITE 900, ALEXANDRIA, VA, 22314, US

(21) Appl. No.: 10/588,846

(22) PCT Filed: Mar. 18, 2005
(86) PCT No.: PCT/AU05/00396

§ 371 Date: Apr. 30, 2007

(30) Foreign Application Priority Data
Mar. 19, 2004 (AU) 2004901473

(51) Int. Cl. C07D 245/04

(52) U.S. Cl. 540/472

(57) ABSTRACT

The present invention provides a method for preparing compounds comprising a plurality of cucurbituril groups. The method comprises forming a mixture comprising one or more compounds of the formula A-L-A wherein L is a linking group and A is group of the formula (A), and an acid, and exposing the mixture to conditions effective for at least some of the groups A to form cucurbituril groups.

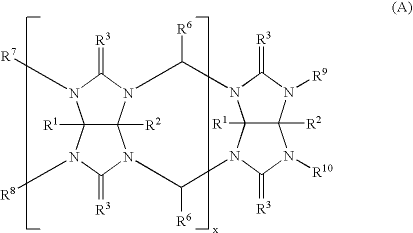

### FIELD OF THE INVENTION

The present invention relates to a method for preparing compounds comprising a plurality of cucurbituril groups.

### BACKGROUND TO THE INVENTION

Cucurbiturils are a class of macrocyclic compounds based on oligomers of glycoluril or glycoluril analogues.

“Cucurbituril” is the name given to the cyclic oligomer formed by linking six (6) glycoluril molecules via methylene bridges. However, the term “cucurbituril” has also been used, and is used in this specification, to refer to a class of compounds. To avoid confusion, the compound cucurbituril is referred to in this specification as “unsubstituted cucurbit[6]uril”.

Unsubstituted cucurbit[6]uril was first described in the literature in 1905 in a paper by R. Behrend, E. Meyer and F. Rusche, Leibigs Ann. Chem., 339, 1, 1905. The macrocyclic structure of unsubstituted cucurbit[6]uril was first described in 1981 by W. A. Freeman et. al., “Cucurbituril”, J. Am. Chem. Soc., 103 (1981), 7367-7368. Unsubstituted cucurbit[6]uril has a chemical formula of C₃₆H₃₆N₂₄O₁₂ and is a macrocyclic compound having a central cavity.

The substituted cucurbituril decamethylcucurbit[5]uril was first synthesised and identified in 1992 by Flinn et. al., Angew. Chem. Int. Ed. Engl., 1992, 31, 1475.

Various unsubstituted and substituted cucurbit[4 to 12]urils and methods for preparing unsubstituted and substituted cucurbit[4 to 12]urils are described in the applicant's international patent application No. PCT/AU00/00412 (WO 00/68232), incorporated herein by reference.

A class of cucurbit[4 to 20]urils and methods for preparing this class of cucurbit[4 to 20]urils are also described in U.S. Pat. No. 6,365,734.

Cucurbit[n]urils comprise a rigid central cavity with two portals to the central cavity. These portals are surrounded by polar groups and are narrower in diameter than the internal diameter of the central cavity.

Various cucurbituril analogues have also recently been described (for example, in Lagona J. et al “Cucurbit[n]uril Analogues”, Organic Letters, 2003, Vol 5, No. 20, 3745-3747). These analogues have a similar macrocyclic structure to cucurbit[n]urils and form complexes with other compounds in a similar manner to cucurbit[n]urils. Like cucurbit[n]urils, cucurbituril analogues comprise a rigid central cavity with two portals to the central cavity, the portals being surrounded by polar groups and having a narrower diameter than the internal diameter of the central cavity.

Cucurbit[4 to 12]urils and cucurbituril analogues selectively complex various molecules. For example, the central cavity of a cucurbit[4 to 12]uril or a cucurbituril analogue selectively encapsulates gases and volatile molecules. Cucurbit[4 to 12]urils and cucurbituril analogues can also selectively form complexes with molecules at the polar ends of the central cavity.

Cucurbit[4 to 12]urils and cucurbituril analogues can be used to form complexes with, and then later release, gases, volatiles, and other molecules. These properties give cucurbit[4 to 12]urils and cucurbituril analogues a wide variety of uses. These uses include for example:
entrapment and removal of pollutants,
use as odourisers, releasing fragrances slowly over time,
to trap unpleasant odours or toxic vapours, and
chemical purification or separation techniques, for example, in chromatographic columns.

These uses of cucurbiturils and cucurbituril analogues involve forming a complex of the cucurbituril or cucurbituril analogue with another molecule. Typically the complex is formed by contacting the cucurbituril or cucurbituril analogue with the molecule by moving a gas or liquid containing the molecule past the cucurbituril or cucurbituril analogue. However, in many cases, some of the cucurbituril or cucurbituril analogue molecules are lost during this process, either by being physically blown or washed away by the movement of the gas or liquid past the cucurbituril or cucurbituril analogue or by the cucurbituril or cucurbituril analogue dissolving in the liquid and being washed away with the liquid.

### SUMMARY OF THE INVENTION

The present inventor has sought to develop a method for preparing compounds comprising multiple (two or more) cucurbituril groups. A compound comprising multiple cucurbituril groups is, due to the size of the compound, generally less susceptible to being physically blown or washed away by the movement of a gas or liquid than a smaller cucurbituril or cucurbituril analogue molecule comprising a single cucurbituril group. Further, because of the high molecular weight of such a compound, if the compound dissolves in a liquid, an artificial or biological membrane or film can be used to retain the compound in a given environment in the liquid.

A compound comprising a plurality of cucurbituril groups can be prepared by preparing cucurbiturils or cucurbituril analogues and then linking the cucurbiturils or cucurbituril analogues using reactions known in the art for linking organic molecules. For example, two cucurbiturils or cucurbituril analogues may be linked by a condensation reaction between appropriate substituents on the cucurbiturils or cucurbituril analogues. However, this process involves the step of first forming the cucurbiturils or cucurbituril analogues and then the separate step of linking the formed cucurbiturils or cucurbituril analogues.

It would be advantageous to provide an alternative method for preparing compounds comprising a plurality of cucurbituril groups.

The present inventor has now found an alternative method for preparing compounds comprising a plurality of cucurbituril groups.

In one aspect, the present invention provides a method for preparing a compound comprising a plurality of cucurbituril groups, the method comprising the steps of:

(a) forming a mixture comprising one or more compounds of the formula (1)
A-L-A (1)

wherein

L is a linking group; and

each A is independently selected and is a group of the formula (A)

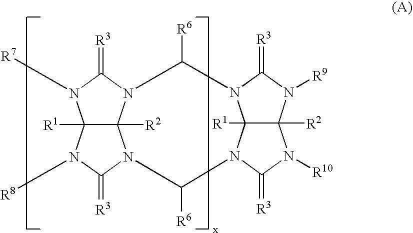

wherein:

for each unit of the formula (B) 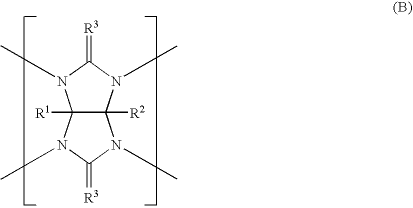
in formula (A),
R¹ and R² may be the same or different, and are each independently selected from a bond with L or
a univalent radical, or
R¹, R² and the carbon atoms to which they are bound together form an optionally substituted cyclic group, or
R¹ of one unit of the formula (B) and R² of an adjacent unit of the formula (B) together form a bond or a divalent radical,
and
each R³ is independently selected from the group consisting of ═O, ═S, ═NR′, ═CXZ, ═CZR′, ═CXR″ and ═CZ₂, wherein Z is an electron withdrawing group, R′ is selected from the group consisting of a bond with L, H, an optionally substituted straight chain, branched or cyclic, saturated or unsaturated hydrocarbon radical, or an optionally substituted heterocyclyl radical, and R″ is a bond with L; and

each R⁶ is independently selected from the group consisting of a bond with L, H, alkyl and aryl;

R⁷ and R⁸ may be the same or different and are independently selected from the group consisting of H and —CHR⁶OR⁶, or R⁷ and R⁸ together form the group —CHR⁶—O—CHR⁶—, where each R⁶ is independently selected from the group consisting of a bond with L, H, alkyl and aryl;

R⁹ and R¹⁰ may be the same or different and are independently selected from the group consisting of H and —CHR⁶OR⁶, or R⁹ and R¹⁰ together form the group —CHR⁶—O—CHR⁶—, where each R⁶ is independently selected from the group consisting of a bond with L, H, alkyl and aryl; and

x is 0 or an integer from 1 to 10, typically x is 0, 1, 2, 3 or 4;

provided that at least one R¹, R² or R⁶ is a bond with L or at least one R³ is ═NR″, ═CZR″ or ═CXR″ where R″ is a bond with L;

and an acid; and

(b) exposing the mixture to conditions effective for at least some of the groups A to react to form cucurbituril groups, thereby forming a compound comprising a plurality of cucurbituril groups.

R¹ and R² may be the same or different in different units of the formula (B) in formula (A).

Z may for example be —NO₂, —CO₂R, —COR or —CX₃, wherein X is halo and R is H, an optionally substituted straight chain, branched or cyclic, saturated or unsaturated hydrocarbon radical or an optionally substituted heterocyclyl radical.

Typically each R⁶ in formula (A) is H, alkyl or aryl, more typically H. Typically R³ is ═O.

In some embodiments, the mixture further comprises one or more compounds capable of linking two groups A (“an Additional Compound”). Typically, in such embodiments, at least some of the cucurbituril groups are formed from a group A of one molecule of the formula (1), a group A of another molecule of the formula (1) and one or more of the Additional Compounds.

The Additional Compound may be a compound of formula (2) or formula (6) as defined below.

Typically, the Additional Compound is a compound of the formula (2):

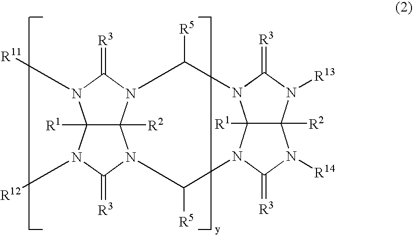

wherein for each unit of the formula (B)

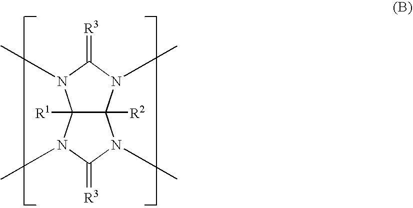

in the compound,

R¹ and R² may be the same or different, and are each a univalent radical, or

R¹, R² and the carbon atoms to which they are bound together form an optionally substituted cyclic group, or

R¹ of one unit of the formula (B) and R² of an adjacent unit of the formula (B) together form a bond or a divalent radical,

and

each R³ is independently selected from the group consisting of ═O, ═S, ═NR, ═CXZ, ═CRZ and ═CZ₂, wherein Z is an electron withdrawing group such as —NO₂, —CO₂R, —COR or —CX₃, X is halo, and R is H, an optionally substituted straight chain, branched or cyclic, saturated or unsaturated hydrocarbon radical, or an optionally substituted heterocyclyl radical;

each R⁵ is independently selected from the group consisting of H, alkyl and aryl;

R¹¹ and R¹² may be the same or different and are independently selected from the group consisting of H and —CHR⁵OR⁵, or R¹¹ and R¹² together form the group —CHR⁵—O—CHR⁵—, where each R⁵ is independently selected and is as defined above,

R¹³ and R¹⁴ may be the same or different and are independently selected from the group consisting of H and —CHR⁵OR⁵, or R¹³ and R¹⁴ together form the group —CHR⁵—O—CHR⁵—, where each R⁵ is independently selected and is as defined as above; and

y is 0 or an integer from 1 to 9; typically y is 0, 1 or 2.

R¹ and R² may be the same or different in different units of the formula (B) in formula (2). Typically R⁵ is H. Typically R³ is ═O.

In some embodiments of the present invention, the Additional Compound is a bis-hydrazine compound of the formula (6):

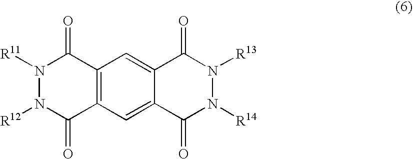

wherein R¹¹, R¹², R¹³ and R¹⁴ are as defined above for formula (2); typically R¹¹ to R¹⁴ are each H.

If the groups A in the compound or compounds of formula (1) in the mixture on average comprise one to two units of the formula (B), the mixture typically comprises one or more Additional Compounds.

Step (b) of the method of the present invention typically comprises heating the mixture to a temperature of from 20° C. to 120° C.

In some embodiments of the present invention, step (b) further comprises contacting the one or more compounds of formula (1) with a compound that can form bridges between groups A, or between a group A and an Additional Compound. Typically the one or more compounds of the formula (1) is contacted with such a compound by incorporating the compound into the mixture comprising the one or more compounds of the formula (1) and the acid.

Typically the compound that can form bridges between groups A, or between a group A and an Additional Compound, is a compound of the formula R⁵COR⁵ where R⁵ is as defined above and each R⁵ is independently selected, a compound of the formula R⁵OC(R⁵)₂OR⁵ where R⁵ is as defined above and each R⁵ is independently selected, trioxane, optionally substituted 3,4-dihydropyran or optionally substituted 2,3-dihydrofuran. These compounds are capable of forming bridges of the formula —CHR⁵— between groups A, and between a group A and a compound of formula (2) or (6). As will be apparent to a person skilled in the art, these compounds form bridges of the formula —CHR⁵— between groups A, and between a group A and a compound of formula (2) or (6), by reaction with, or replacement of, the groups R⁷ to R¹⁴ to form bridges of the formula —CHR⁵— bound to the nitrogen atoms to which R⁷ to R¹⁴ were bound.

As will be apparent to a person skilled in the art, in some embodiments of the invention, it is not necessary to include a compound that can form bridges between groups A, or between a group A and an Additional Compound, in the mixture in order to form cucurbituril groups in step (b) of the method of the present invention. For example, if all the groups R⁷, R⁸, R⁹, R¹⁰, R¹¹, R¹², R¹³ and R¹⁴ in the compounds of formula (1) and the Additional Compounds of formula (2) or (6), if any, in the mixture are other than H, the groups A can react with each other and with the Additional Compounds of formula (2) and (6), if any, in step (b) of the method of the present invention to form cucurbituril groups without the presence of such a compound. However, if all of the groups R⁷, R⁸, R⁹, R¹⁰, R¹¹, R¹², R¹³ and R¹⁴ are H, then such a compound must be included in the mixture in order for the groups A to react with each other and with the Additional Compounds of formula (2) or (6), if any, to form cucurbituril groups in step (b) of the method.

Typically, if the molar ratio of the groups R⁷, R⁸, R⁹, R¹⁰, R¹¹, R¹², R¹³ and R¹⁴ in the compounds of formula (1) , (2) and (6) in the mixture which are H to those which are not H is greater than 1, then a compound that can form bridges between groups A, and between a group A and a compound of formula (2) or (6), is included in the mixture.

The mixture comprising the one or more compounds of formula (1) and the acid may be prepared by adding the one or more compounds of the formula (1) to the acid and mixing. If the mixture further comprises other components, such as one or more Additional Compounds or one or more compounds that can form bridges between groups A, or between a group A and an Additional Compound, the mixture may be prepared by combining the various components of the mixture in any order.

In formula (1), the linking group L can be any group capable of linking two groups A. L is typically a divalent group linking two groups A. However, in some embodiments, the group L may have more than one bond to one or both of the groups A. The linking group may for example be an organic group such as a hydrocarbon chain or a polymer chain, or a metal or metal complex. L is typically a polymer or other organic group. The linking group L can be as short as —CH₂—, —O— or —NH—, or as long as a polymer chain.

Typically the group A is bound to the linking group L via a bond at one R¹ or R² in the group A (i.e. one R¹ or R² in formula (A) is a bond with L), or by a bond at both R¹ and R² in one unit of the formula (B) in the group A (i.e. both R¹ and R² in one unit of the formula (B) in formula (A) are each a bond with L). However, in some embodiments of the present invention, the group A is bound to the linking group L by a bond at R⁶ or R³ (i.e. one R⁶ in formula (A) is a bond with L, or one R³ in formula (A) is ═NR″, ═CXR″ or ═CZR″ where R″ is a bond with L).

In some embodiments, the linking group L comprises one or more further groups A. For example, the group L may comprise a polymer chain which is substituted by one or more groups containing groups of the formula (A).

In another aspect, the present invention provides a compound comprising a plurality of cucurbituril groups prepared by the method of the present invention.

### DEFINITIONS

Having regard to the cucurbiturils described in WO 00/68232 and U.S. Pat. No. 6,365,734 and the inventor's further work, the class of cucurbiturils is broader than that described in either of WO 00/68232 or U.S. Pat. No. 6,365,734.

As used herein, the term “cucurbituril” refers to a compound of the formula (C):

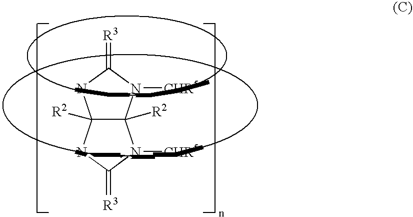

wherein:

for each unit of the formula (D):

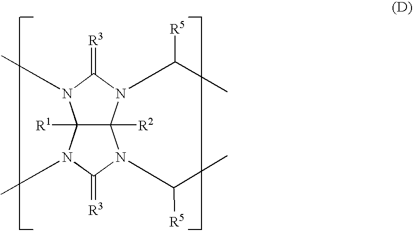

in the compound,

R¹ and R² may be the same or different, and are each a univalent radical, or

R¹, R² and the carbon atoms to which they are bound together form an optionally substituted cyclic group, or

R¹ of one unit of the formula (D) and R² of an adjacent unit of the formula (D) together form a bond or a divalent radical,

each R³ is independently selected from the group consisting of ═O, ═S, ═NR, ═CXZ, ═CRZ, and ═CZ₂, wherein Z is an electron withdrawing group such as —NO₂, —CO₂R, —COR or —CX₃, X is halo and R is H, an optionally substituted straight chain, branched or cyclic, saturated or unsaturated hydrocarbon radical, or an optionally substituted heterocyclyl radical, and

each R⁵ is independently selected from the group consisting of H, alkyl and aryl;

and n is the degree of polymerisation, that is, the number of units of the formula (D) in the compound.

To differentiate various cucurbiturils, the inventors have adopted the term “cucurbit[n]uril”, where n is the degree of polymerisation of the cucurbituril, that is, the number of units of the formula (D) in the macrocyclic ring of the cucurbituril. For example, a cucurbituril comprising eight units of the formula (D) joined together would be denoted as cucurbit[8]uril.

Unless otherwise specified, the terms “cucurbituril” and “cucurbit[n]uril” as used herein refer to a cucurbit[n]uril where n is an integer from 4 to 12.

As used herein, the terms “unsubstituted cucurbituril” and “unsubstituted cucurbit[n]uril” refer to a cucurbituril in which R³ is ═O, and R¹, R² and R⁵ are H, in all the units of formula (D) in the cucurbituril. As used herein, the terms “substituted cucurbituril” and “substituted cucurbit[n]uril” refer to a cucurbituril other than an unsubstituted cucurbituril.

As used herein, the term “cucurbituril analogue” refers to a compound comprising a macrocyclic ring similar to the macrocyclic ring of a cucurbit[n]uril such that the macrocyclic ring comprises a rigid central cavity with two portals to the central cavity, the portals being surrounded by polar groups and having a narrower diameter than the internal diameter of the central cavity, and wherein the compound is capable of forming complexes with other molecules in the same or substantially the same manner as a cucurbit[n]uril. A cucurbituril analogue may for example have the basic cyclic structure of a cucurbituril of the formula (C) as defined above but in which one or some, but not all, of the units of the formula (D) are replaced by another group such as a group of the formula:

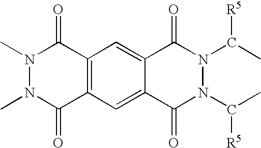

As used herein, the term “cucurbituril group” refers to the macrocyclic ring of a cucurbituril or cucurbituril analogue, the macrocyclic ring comprising a rigid central cavity with two portals to the central cavity, the portals being surrounded by polar groups and having a narrower diameter than the internal diameter of the central cavity.

As used herein, the term “cucurbit[n]uril group” refers to a cucurbituril group having the cyclic structure shown in formula (C) above, that is, that part of formula (C) excluding the groups R¹, R², R⁵ and R.

As used herein, the term “glycoluril analogue” refers to a compound of the formula (5):

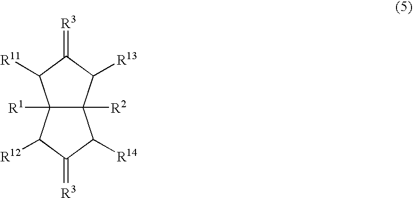

wherein

R¹ and R² may be the same or different, and are each a univalent radical, or

R¹, R² and the carbon atoms to which they are bound together form an optionally substituted cyclic group, and R³ and R¹¹ to R¹⁴ are as defined above for formula (2).

As used herein, the term “alkyl” refers to a straight chain, branched or mono- or poly-cyclic alkyl. Typically the alkyl is a C₁ to C₃₀ alkyl, for example, a C₁ to C₆ alkyl. Examples of straight chain and branched alkyl include methyl, ethyl, propyl, isopropyl, butyl, isobutyl, sec-butyl, tert-butyl, pentyl, iospentyl, sec-pentyl, 1,2-dimethylpropyl, 1,1-dimethylpropyl, hexyl, 4-methylpentyl, 1-methylpentyl, 2-methylpentyl, 3-methylpentyl, 1,1-dimethylbutyl, 2,2-dimethylbutyl, 3,3-dimethylbutyl, 1,2-dimethylbutyl, 1,3-dimethylbutyl, 1,2,2-trimethylpropyl and 1,1,2-trimethylpropyl. Examples of cyclic alkyl include cyclopropyl, cyclobutyl, cyclopentyl and cyclohexyl.

As used herein, the term “alkenyl” refers to a straight chain, branched or cyclic alkenyl. Typically the alkenyl is a C₂ to C₃₀ alkenyl, for example a C₂ to C₆ alkenyl. Examples of alkenyl include vinyl, allyl, 1-methylvinyl, butenyl, isobutenyl, 3-methyl-2-butenyl, 1-pentenyl, cyclopentenyl, 1-methylcyclopentenyl, 1-hexenyl, 3-hexenyl, cyclohexenyl, 1-heptenyl, 3-heptenyl, 1-octenyl, cyclooctenyl, 1-nonenyl, 2-nonenyl, 3-nonenyl, 1-decenyl, 3-decenyl, 1,3-butadienyl, 1,4-pentadienyl, 1,3-cyclopentadienyl, 1,3-hexadienyl, 1,4-hexadienyl, 1,3-cyclohexadienyl, 1,4-cyclohexadienyl, 1,3-cycloheptadienyl, 1,3,5-cycloheptatrienyl and 1,3,5,7-cyclooctatetraenyl.

As used herein, the term “alkynyl” refers to a straight chain, branched or cyclic alkynyl. Typically the alkynyl is a C₂ to C₃₀ alkynyl, for example, a C₂ to C₆ alkynyl.

As used herein, the term “aryl” refers to a radical of a single, polynuclear, conjugated or fused aromatic hydrocarbon or aromatic heterocyclic ring system. Examples of aryl include phenyl, naphthyl and furyl. When the aryl comprises a heterocyclic aromatic ring system, the heterocyclic aromatic ring system may contain 1 to 4 heteroatoms independently selected from N, O and S.

### DETAILED DESCRIPTION OF PREFERRED EMBODIMENTS

As will be apparent to a person skilled in the art, in formulas (1) and (2), R¹ and R² can be any group that does not prevent the groups A in the one or more compounds of formula (1) reacting to form cucurbituril groups in step (b) of the method of the present invention. The present invention is not limited to methods where R¹ and R² are particular groups.

In formulas (1), (2) and (5), when R¹ or R² is a univalent radical, the univalent radical is typically —R, —OR, —NR₂ where each R is independently selected, —NO₂, —CN, —X, —COR, —COX, —COOR, 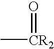
where each R is independently selected, 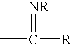
where each R is independently selected, —SeR, —SiR₃ where each R is independently selected, —SR, —SOR, 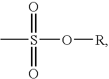
—SO₂R, —S—S—R, —BR₂ where each R is independently selected, —PR₂ where each R is independently selected, 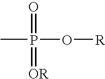
where each R is independently selected, 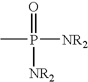
where each R is independently selected, —P⁺R₂ where each R is independently selected, or a metal or metal complex,

wherein R is H, an optionally substituted straight chain, branched or cyclic, saturated or unsaturated hydrocarbon radical, or an optionally substituted heterocyclyl radical, and X is halo.

When R¹ or R² is a univalent radical, the univalent radical may for example be H, an optionally substituted alkyl (e.g. methyl, ethyl, propyl, isopropyl, n-butyl, sec-butyl, isobutyl, tert-butyl, etc), optionally substituted alkenyl, optionally substituted alkynyl, optionally substituted heterocyclyl or optionally substituted aryl (e.g. phenyl, naphthyl, pyridyl, furanyl, thiophenyl or pyrazolyl), —OR, —SR or —NR₂.

In some embodiments, R¹ or R² is a univalent radical comprising less than 30 carbon atoms. The univalent radical may for example be a C₁ to C₃₀ alkyl, C₂ to C₃₀ alkenyl, a cyclic hydrocarbon group comprising 5 to 30 carbon atoms, an aliphatic cyclic group comprising 4 to 30 carbon atoms with one or more heteroatoms such as O, N or S, an aryl group comprising 6 to 30 carbon atoms and no heteroatoms, or a heteroaryl group comprising 5 to 30 carbon atoms with one or more hetero atoms such as O, N or S.

R¹ or R² may for example be an alkoxy group such as methoxy, ethoxy, propyloxy etc. R¹ or R² may also be a hydroxy, halo, cyano, nitro, amino, alkylamino or alkylthio radical.

Examples of optionally substituted cyclic groups formed by R¹, R² and the carbon atoms to which they are bound, include optionally substituted saturated or unsaturated cyclic hydrocarbon groups comprising 5 to 30 carbon atoms, and optionally substituted saturated or unsaturated cyclic groups comprising 3 to 30, typically 4 to 30, carbon atoms with one or more heteroatoms such as O, N or S. The optionally substituted cyclic group may comprise two or more fused rings.

The divalent radical which may link R¹ and R² of adjacent units of the formula (B) in formula (A), adjacent units of the formula (B) in formula (2), or adjacent units of the formula (D) in a cucurbit[n]uril, may, for example, be a divalent optionally substituted straight chain or branched, saturated or unsaturated hydrocarbon radical comprising 1 or more carbon atoms. The divalent radical may consist of or contain one or more heteroatoms such as O, N or S.

When R or R′ is an optionally substituted hydrocarbon radical or an optionally substituted heterocyclyl radical, R or R′ may, for example, be an optionally substituted alkyl, an optionally substituted alkenyl, an optionally substituted alkynyl or an optionally substituted aryl.

When R or R′ is an optionally substituted hydrocarbon radical or an optionally substituted heterocyclyl radical, the hydrocarbon radical or the heterocyclyl radical may be substituted by one or more substituents. Similarly, when R¹, R² and the carbon atoms to which they are bound together form an optionally substituted cyclic group, the cyclic group may be substituted by one or more substituents. The optional substituents can be any group and may for example be an optionally substituted alkyl, an optionally substituted alkenyl, an optionally substituted alkynyl, an optionally substituted heterocyclyl, an optionally substituted aryl, halo (F, Cl, Br or I), hydroxyl, alkoxyl, carbonyl, acyl halide, nitro, carboxylic acid, carboxylic acid ester, amino, imino, cyano, isocyanate, thiol, thiol-ester, thio-amide, thio-urea, sulfone, sulfide, sulfoxide or sulfonic acid group or a metal or metal complex. The optional substituent may also be a borane, a phosphorous containing group such as a phosphine, alkyl phosphine, phosphate or phosphoramide, a silicon containing group or a selenium containing group.

Typically Z is —NO₂, —CO₂R, —COR or —CX₃, where X is halo (F, Cl, Br or I) and R is H, alkyl, alkenyl, alkynyl, aryl, heteroaryl or saturated or unsaturated heterocyclyl.

In some embodiments of the present invention, R³ is ═O and R⁵ is H in all the units of formula (B) in the compounds of formulas (1) and (2).

The group L may be any group capable of linking two groups A. L is typically a divalent organic group. In some embodiments L is a polymer. In some embodiments L is a group of the formula —(CR₂)ₐ-(E-(CR₂)_{b}—)_{c}(CR₂)_{d}— or —(CR₂)ₐ-(E-(CR═CR)_{b}—)_{c}(CR₂)_{d}—

wherein:

E is —O—, —NR—, —S—, a saturated or unsaturated divalent hydrocarbon radical, or an optionally substituted aliphatic or aromatic divalent heterocyclyl group;

R is as defined above for formula (2); and

a, b, c and d are each 0 or an integer from 1 to 30, provided that not all of a, b, c and d are 0.

For example, L may be a group of the formula —(CR₂)ₐ—, —(CR═CR)ₐ— or —(NR)ₐ—

where R is as defined above, and a is an integer from 1 to 30. When E is an optionally substituted heterocyclyl radical, the heterocyclyl radical may be optionally substituted by one or more substituents. The optional substituents can be any group and may for example be an optionally substituted alkyl, an optionally substituted alkenyl, an optionally substituted alkynyl, an optionally substituted heterocyclyl, an optionally substituted aryl, halo (F, Cl, Br or I), hydroxyl, alkoxyl, carbonyl, acyl halide, nitro, carboxylic acid, carboxylic acid ester, amino, imino, cyano, isocyanate, thiol, thiol-ester, thio-amide, thio-urea, sulfone, sulfide, sulfoxide or sulfonic acid group or a metal or metal complex. The optional substituent may also be a borane, a phosphorous containing group such as a phosphine, alkyl phosphine, phosphate or phosphoramide, a silicon containing group or a selenium containing group.

L may, for example, be —CH₂—, —(CH₂)ₙ—, —(CH═CH)ₙ—, —O—, —NH—, —CH₂—NH—, —CH(CH₃)(CH₂)ₙCH(CH₃)— or

—(CH₂)ₙ—N(CH₃)CH₂CH₂N(CH₃)—(CH₂)ₚ—,

where n and p are an integer, for example an integer from 1 to 30, such as 1, 2, 3, 4, 5, 6, 7 etc. L may also be an organometallic group such as

—CH₂Si(R)₂CH₂— where R is H, an optionally substituted straight chain, branched or cyclic, saturated or unsaturated hydrocarbon radical or an optionally substituted heterocyclyl radical. In some embodiments, L is, or comprises, a metal atom and the compound of the formula (1) is a metal complex.

In preferred embodiments, the Additional Compound is a compound of formula (2). As will be apparent to a person skilled in the art, if the groups R⁷, R⁸, R⁹, R¹⁰, R¹¹, R¹², R¹³ and R¹⁴ in the compounds of formula (1) and (2) in the mixture are other than hydrogen, the groups A can react with each other and with the compounds of formula (2) to form cucurbituril groups in step (b) of the method without the presence of a compound that can form bridges between groups A, and between a group A and a compound of formula (2). However, if all of these groups are H, or if, in the groups A and the compound or compounds of formula (2) that are to form the cucurbituril group, the total number of these groups which are H is greater than the total number which are not H, then a compound that can form bridges between groups A, and between a group A and a compound of formula (2), must be included in the mixture in order for the groups A, and the one or more compounds of formula (2), to form the cucurbituril group in step (b) of the method.

The compound that can form bridges between groups A, and between a group A and a compound of formula (2) or (6), is typically selected from the group consisting of compounds of the formula R⁵COR⁵ wherein each R⁵ is independently selected from the group consisting of H, alkyl and aryl, compounds of the formula R⁵OC(R⁵)₂OR⁵ wherein each R⁵ is independently selected from the group consisting of H, alkyl and aryl, trioxane, optionally substituted 3,4-dihydropyran and optionally substituted 2,3-dihydrofuran. The optionally substituted 3,4-dihydropyran or optionally substituted 2,3-dihydrofuran may be substituted by groups such as alkyl, alkenyl, alkynyl, aryl or halo. The compound of the formula R⁵COR⁵ may, for example, be formaldehyde.

Typically the mixture further comprises a templating compound. As used herein, the term “templating compound” refers to a compound that affects the relative amount of different sized cucurbituril groups formed in the method of the present invention. For example a templating compound when added to the mixture, may alter the ratio of, say, cucurbit[5]uril groups to cucurbit[6]uril groups, when that ratio is compared with that ratio of cucurbit[5]uril groups to cucurbit[6]uril groups that is formed using a mixture not containing a templating compound or containing a different templating compound, but otherwise reacted under identical conditions.

Typically, the templating compound is a salt. However, it has been found that many other compounds can also act as a templating compound.

Any compound that can alter the ratio of different sized cucurbituril groups formed in the method of the present invention can be used as the templating compound. The templating compound may be an organic compound, a salt of an organic compound, or an inorganic compound. Suitable compounds that may be used as a templating compound include ammonium chloride, lithium chloride, sodium chloride, potassium chloride, rubidium chloride, caesium chloride, ammonium bromide, lithium bromide, sodium bromide, potassium bromide, rubidium bromide, caesium bromide, lithium iodide, sodium iodide, potassium iodide, rubidium iodide, caesium iodide, potassium sulfate, lithium sulfate, tetrabutylammonium chloride, tetraethylammonium chloride, o-carborane, thioacetamide, N-(1-napthyl) ethylenediamine, 2,2′-biquinoline, p-bromoanaline, taurine, blue tetrazolium, 2-amino-3-methyl benzoic acid, indol-3-aldehyde, cystine, 4-acetamidoaniline, p-aminophenol, acetamide, 4-aminoacetophenone, 4-dimethylaminobezaldehyde, 2-aminobenzimadazol, bis-(4,4′-bipyridyl)-α, α′-xylene, red phosphorus, and lithium p-toluenesulfonate. The present inventor believes that a large number of other compounds could be suitable for use as templating compounds and therefore the above list should not be considered to be exhaustive. The anions of the acid may also be considered to be a templating compound.

The templating compounds may be added singly to the reaction mixture, or two or more templating compounds may be added to the reaction mixture.

If a salt is used as the templating compound, the salt is preferably a metal halide, ammonium halide, metal sulphate or metal tosylate. It is preferred that the anion of the salt corresponds to the anion of the acid used. For example, where the acid used is hydrochloric acid, a metal chloride or ammonium chloride is a preferred salt. Similarly, iodide-containing salts are preferably used where hydriodic acid is the acid, and bromide-containing salts are preferably used where hydrobromic acid is used.

The acid is preferably a strong mineral acid or a strong organic acid. In principle, any acid can be used. The acid acts to catalyse the reactions taking place.

Preferred acids include sulfuric acid, hydrochloric acid, hydrobromic acid, hydriodic acid, deuterated sulfuric acid, phosphoric acid, p-toluenesulfonic acid, and methane sulphonic acid. It will be appreciated that this list is not exhaustive and that any acid that can catalyse the reaction may be used in the method of the present invention.

The mixture may or may not be an aqueous system. When the mixture is an aqueous system, the acid is preferably included in the mixture in an amount such that the concentration of the acid in the mixture is greater than 5M.

A solvent may also be added to the mixture. The solvent may for example be selected from trifluoroacetic acid, methanesulfonic acid, 1,1,1-trifluorethanol or an ionic liquid.

Typically, step (b) comprises exposing the mixture to conditions effective for at least some of the groups A to react to form cucurbituril groups, wherein at least some of the cucurbituril groups formed are formed from a group A of one molecule of the formula (1) and a group A of another molecule of the formula (1).

Step (b) of the method of the present invention typically comprises heating the mixture to a temperature of from 20° C. to 120° C. for a period of time sufficient to form a compound comprising a plurality of cucurbituril groups. Typically the temperature is 60° C. to 110° C., most preferably from 80° C. to 110° C. It is preferred that boiling of the mixture is avoided. Heating under reflux is not required but may be used.

In some embodiments, step (b) comprises contacting the one or more compounds of the formula (1) with a compound that can form bridges between groups A, or between a group A and an Additional Compound, and heating the mixture to a temperature of from 20° C. to 120° C. for a period of time sufficient to form a compound comprising a plurality of cucurbituril groups.

The method of the present invention is described below by reference to the following non-limiting examples.

1. Preparation of Compounds of Formula (1) and (2)

Compounds of formula (1) and (2) may be prepared by a variety of methods.

(a) Synthesis of Glycoluril Analogues of Formula (5)

Formula (2) encompasses glycoluril analogues of the formula (5) as defined above.

Formula (5) encompasses glycoluril of the formula:

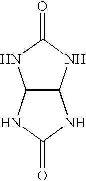

Formula (5) also encompasses substituted glycolurils of the formula:

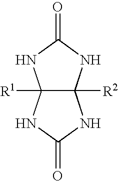

There are a large number of substituted glycolurils known in the literature. Particular reference is made to the review article by Harro Petersen in Synthesis, 1973, 249-293, which contains a list of about 30 substituted glycolurils. The literature since that article has disclosed several other examples of substituted glycolurils and it is believed that essentially any α- or β-diketone could be used to make a substituted or unsubstituted glycoluril.

Further substituted glycolurils are disclosed in WO 00/68232.

Substituted glycolurils and other glycoluril analogues can be prepared by methods known in the art. For example, substituted glycolurils and other glycoluril analogues can be prepared as described in the review article by Harro Petersen in Synthesis, 1973, 249-293.

Glycoluril analogues can for example be prepared as described in the following reaction schemes:

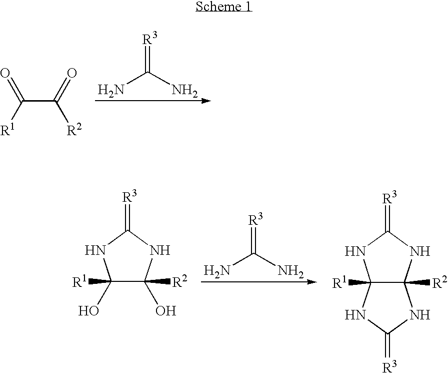

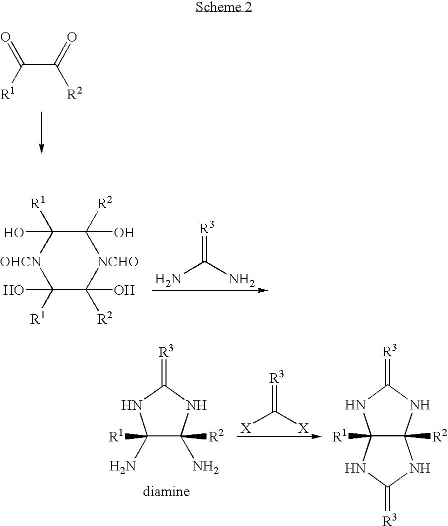

wherein each R¹, R² and R³ group is independently selected and is as defined above for formula (2), and X is a leaving group such as a halo or a thioether.

The reactions of Scheme 1 may be carried out under the following conditions:
a) Reaction in water at room temperature for several days;
b) Reaction in acidic water with or without a cosolvent;
c) Reaction in a hydrocarbon solvent in the presence of an acid catalyst while water of reaction is removed azeotropically;
d) Reaction in a hydrocarbon solvent in the presence of a Lewis acid with or without removal of water generated during reaction.

In Scheme 2, the reaction to form the diamine intermediate is carried out in acid water with or without a cosolvent. The reaction of the diamine intermediate to form the glycoluril analogue is carried out under basic conditions. An example of the reaction of Scheme 2 to form a glycoluril analogue where one R³ is ═NH and the other R³ is ═O is described in I. J. Dagley and M. Kony, Heterocycles 1994, 38, 595.

Scheme 1 can, for example, be used to prepare the following substituted glycolurils (compounds 1.1 to 1.13):

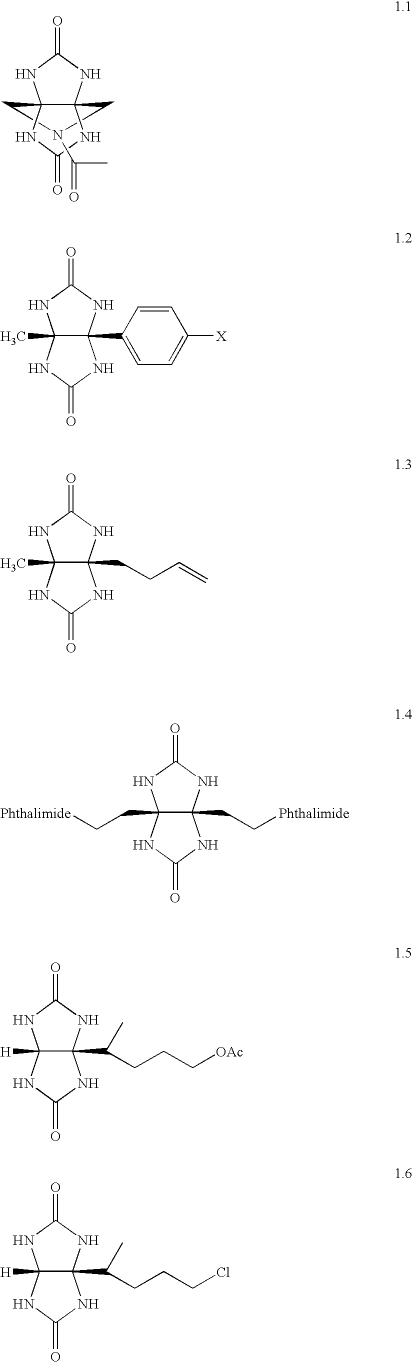

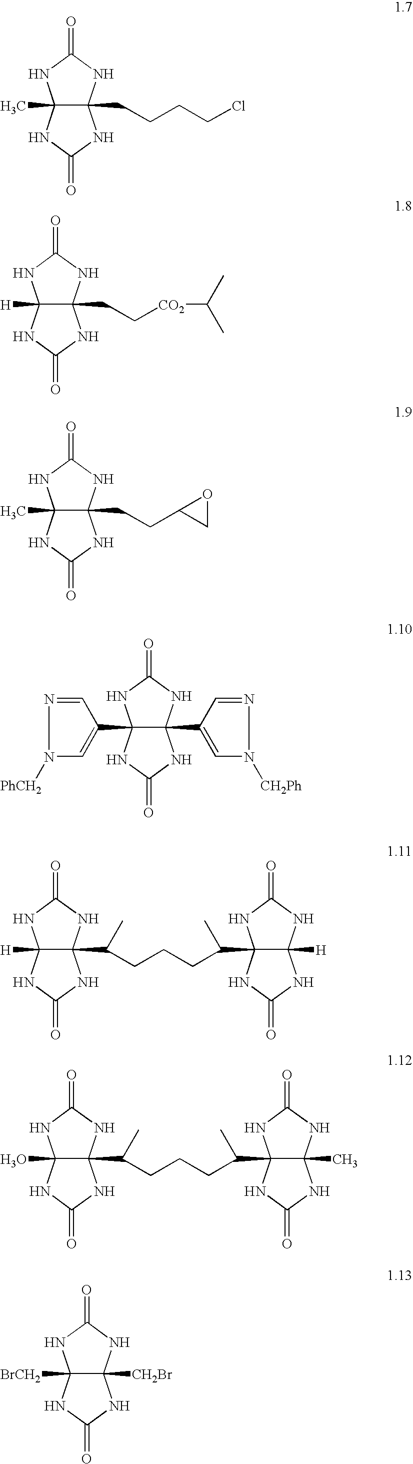

In compound 1.2 above, X is halo.

Compounds of formula (2) in which R¹¹ and R¹² together form the group —CHR⁵—O—CHR⁵— and R¹³ and R¹⁴ together form the group —CHR⁵—O—CHR⁵—, can be prepared by mixing a compound of formula (2) in which R¹¹ to R¹⁴ are H, with trioxane, a compound of the formula (R⁵)₂CO or a compound of the formula R⁵OC(R⁵)₂OR⁵, where R⁵ is as defined above and each R⁵ is independently selected, and an acid, and heating the mixture to about 20° C. to 60° C. Typically, when a strong mineral acid or strong organic acid is used the mixture heated to between 20° C. to 40° C. However, if a weaker acid such as trifluroacetic acid is used, the mixture can be heated to about 60° C.

Compounds of the formula (2) in which some or all of R¹¹ to R¹⁴ are —CHR⁵OR⁵ can be prepared by reacting a compound of formula (2) in which R¹¹ to R¹⁴ are H with a compound of the formula XHR⁵COR⁵, where X is halo and each R⁵ is independently selected and is as defined above, under basic conditions. The reaction typically occurs at room temperature, but the reaction mixture can be heated to about 40° C.

### EXAMPLE 1

To 3a-(4-(1-chlorobutane)-6a-methylglycoluril (compound 1.7) (1 g, 4.2 mmol) suspended in 7M hydrochloric acid (1.27 mL) was added 40% formaldehyde (15 mL) and the mixture stirred at room temperature for 18 h. The resultant precipitated diether was collected by filtration washed with water and dried.

### EXAMPLE 2

To 3a-(p-iodophenyl)-6a-methylglycoluril (compound 1.2 where X=I) (1 g, 1.8 mmol) dissolved in concentrated sulfuric acid (7 mL) was added 40% formaldehyde (1.7 mL) at room temperature. After 20-30 min the mixture was poured into ice water and the precipitated diether was collected by filtration and dried at 80° C. in vacuo, yield 80%.

### EXAMPLE 3

To 3a,6a-diphenylglycoluril (1 g, 1.8 mmol) dissolved in concentrated sulfuric acid (7 mL) was added 40% formaldehyde (0.7 mL) at room temperature. After 20-30 min the mixture was poured into ice water and the precipitated diether was collected by filtration and dried at 80° C. in vacuo, yield 95%.

### EXAMPLE 4

To 3a,6a-di(p-iodophenyl)glycoluril (1 g, 1.8 mmol) dissolved in concentrated sulfuric acid (6 mL) was added 40% formaldehyde (0.54 mL) at room temperature. After 20-30 min the mixture was poured into ice water and the precipitated diether was collected by filtration and dried at 80° C. in vacuo.

### EXAMPLE 5

3a,6a-cyclopentanoglycoluril (1 g, 5.49 mmol) was added to a mixture of dimethylsufoxide (1 mL), water (2 mL) and 40% formaldehyde (1.6 mL) at room temperature and the pH of the mixture adjusted to 9 with 1 M NaOH. After 12 h the mixture was poured into methanol (15 mL) and the precipitated tetrol (compound 2.6) was collected by filtration and dried at 80° C. in vacuo 82% yield.

### EXAMPLE 6

To 3a-(4-but-2-ene)-6a-methylglycoluril (compound 1.3) (1 g, 0.48 mmol) dissolved in trifluoroacetic acid (2 mL) was added 40% formaldehyde (1.46 mL) and the mixture heated to 60° C. for 12 h. Evaporation of the solvent afforded the diether, yield 70%. At short reaction times of less than 1 hr a mixture of alcohols and ethers is formed.

Diether analogues of glycoluril can also be prepared under anhydrous conditions, similar to the method of A. Wu, A. Chakraborty, D. Witt, J. Lagona, F. Damkai, M. A. Ofori, J. K. Chiles, J. C. Fettinger, and L. Isaacs J. Org. Chem. 2002, 67, 5817-5830, incorporated herein by reference.

(b) Synthesis of Oligomers

Compounds of the formula (2) include oligomers comprising 2 to 10 units of formula (B) linked by bridges of the formula —CHR⁵—. Such oligomers, and oligomers of the formula (2) as defined above but in which y is 10, can be used as precursors to prepare compounds of formula (1) as described below at “(c) Synthesis of Compounds of Formula (1)”.

Glycoluril analogues can be used to prepare such oligomers. The oligomers can be prepared by mixing one or more glycoluril analogues with an acid, and if required a compound that can form bridges of the formula —CHR⁵— between glycoluril analogues, and heating the mixture. The compound that can form bridges of the formula —CHR⁵— may be trioxane, a compound of the formula R⁵COR⁵ or a compound of the formula R⁵OC (R⁵)₂OR⁵, wherein R⁵ is as defined above and each R⁵ is independently selected.

Compounds of formula (2) comprising 2 or more units of the formula (B) linked by bridges of the formula —CHR⁵— can also be reacted with a glycoluril analogue or another compound of formula (2) comprising 2 or more units of the formula (B) linked by bridges of the formula —CHR⁵—, under similar conditions to those described above to produce an oligomer containing a greater number of units of the formula (B).

The inventor has found that using suitable reaction temperatures and reaction times, oligomers comprising 2 to 11 units of the formula (B) linked by bridges of the formula —CHR⁵— can be prepared without the oligomers reacting to form cucurbiturils. Typically the oligomers are prepared by heating the reaction mixture to a temperature below 50° C. for a period of less than about 20 hours. This process typically results in the production of a mixture of oligomers comprising different numbers of units of the formula (B). If desired, an oligomer having a particular length may be separated from the other oligomers in the mixture by crystallisation or chromatography.

Examples of compounds of formula (2) include compounds 2.1 to 2.7 having the structure set out below:

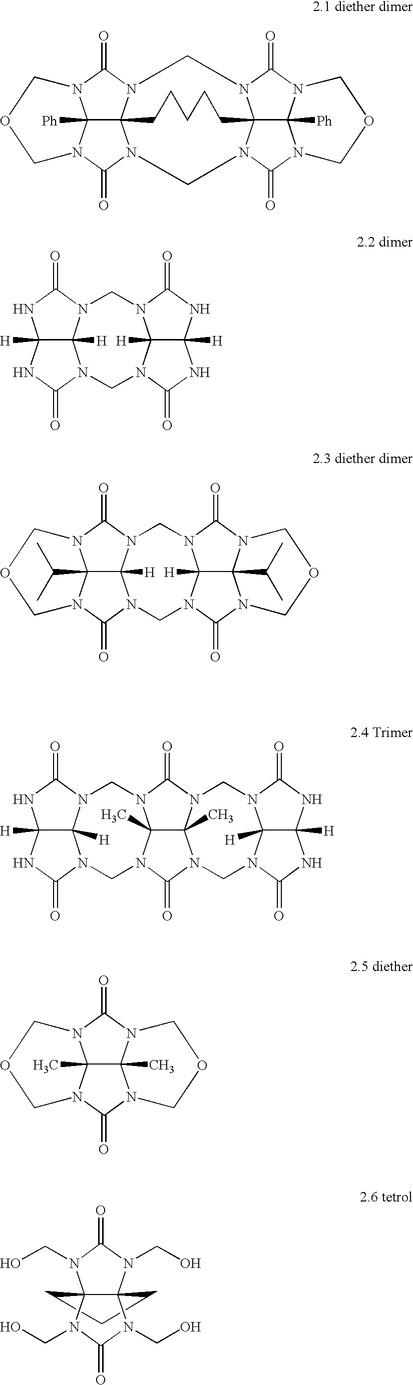

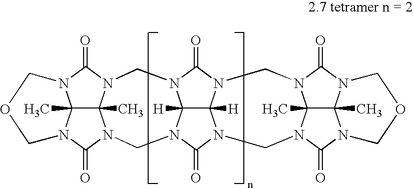

### EXAMPLE 7

To 3a-isopropylglycoluril (1 g, 5.4 mmol) dissolved in trifluoroacetic acid (15 mL) was added 40% formaldehyde (1.62 mL) and the mixture heated at 50° C. or reflux for 24 h. The solvent was evaporated to give predominantly the dimer (compound 2.3) which was purified or used crude.

### EXAMPLE 8

To alkyltethered bisglycoluril (compound 1.11) (500 g, 1.9 mmol) dissolved in trifluoroacetic acid (15 mL) was added 40% formaldehyde (0.855 mL) and the mixture heated at 50° C. or reflux for 24 h. The solvent was evaporated to give predominantly the dimer which was purified or used crude.

### EXAMPLE 9

To 3a-(4-(1-chloro-4-methylbutane)glycoluril (compound 1.6) (1 g, 4.2 mmol) dissolved in trifluoroacetic acid (15 mL) was added 40% formaldehyde (1.26 mL) and the mixture heated at 50° C. or reflux for 24 h. The solvent was evaporated to give the dimer formaldehyde derivative as an ether.

### EXAMPLE 10

The formaldehyde diether derivative of 3a-(4-(1-chloro-4-methylbutane)glycoluril (compound 1.6) (1 g, 2.9 mmol) and the unsubstituted glycoluril dimer (compound 2.2) (1.8 g, 5.8 mmol) were mixed together in concentrated HCl (5 mL) at room temperature. After 30 mins and up to 1 hr the homogeneous mixture was poured into MeOH (10 mL) and the precipitate collected and dried to give predominantly the pentamer.

### EXAMPLE 11

The formaldehyde diether derivative of 3a,6a-diphenylglycoluril (2.9 g, 7.7 mmol), the unsubstituted glycoluril dimer (compound 2.2) (4.7 g, 15.4 mmol) and K₂CO₃ (530 mg) were mixed together in methanesulfonic acid (40 mL) at room temperature, 20 min then 10 min at 50° C. The homogeneous mixture was poured into MeOH (60 mL) and the precipitate collected and dried to give predominantly the pentamer.

### EXAMPLE 12

The formaldehyde diether derivative (compound 2.5) of dimethylglycoluril (1 g, 5.9 mmol) and unsubstituted glycoluril dimer (compound 2.2) (0.91 g, 2.95 mmol) in conc. HCl (2 mL) were stirred together at room temperature for 30 min to 1 hr and the homogeneous mixture was poured into MeOH (10 mL) and the precipitate collected and dried to give predominantly the diether tetramer.

### EXAMPLE 13

The tetrol derivative (compound 2.6) of 3a,6a-cyclopentanoglycoluril (1 g, 3.3mmol) was added to a solution of unsubstituted glycoluril (937 mg,6.6 mmol) in conc. HCl (2 mL) and the mixture stirred at room temperature for 30 min. The homogeneous mixture was poured into MeOH (10 mL) and the precipitate collected and dried to give predominantly the trimer.

Although Examples 7 to 13 concern the preparation of oligomers in which R³ is ═O and R⁵ is H in all the units of formula (B) in the oligomer, analogous processes can be used to prepare oligomers where some or all of the R³ groups are other than ═O and/or some or all of the R⁵ groups are other than H. For example, oligomers where R³ is other than ═O can be prepared using glycoluril analogues where R³ is other than ═O as a starting material. oligomers where R⁵ is other than H can be prepared using analogous processes to those exemplified in Examples 7 to 9 in which a compound of the formula R⁵COR⁵ where one or both R⁵ groups is other than H is used instead of formaldehyde.

(c) Synthesis of Compounds of Formula (1)

Compounds of formula (2), and oligomers of formula (2) as defined above but in which y is 10, can be used as precursors to prepare compounds of formula (1). This is possible through a variety of reactions such as nucleophilic or electrophilic substitution as single or paired electrons, coupling reactions and condensation reactions. Such reactions can, for example be used to prepare compounds such as compounds 3.1 to 3.6 referred to below. Similarly, conventional co-ordination chemistry techniques can also be used to prepare compounds of the formula (1) in which L is a metal or comprises a metal and the compound of formula (1) is a metal complex. Such techniques can for example be used to prepare compounds such as the bis-phenanthroline glycoluril cobalt co-ordination complex 3.7 referred to below.

Alternatively linked glycolurils of formula (1) can be prepared directly from a polyketone (eg R₁COCOR₂COCOR₃ to give compound 1.11 when R₂ is —CHCH₃CH₂CH₂CH₂CH₃CH—, and R₁ and R₃ are CH₃, or compound 1.12 when R₂ is —CH₂CH₂CH₂CH₂CH₂—, and R₁ and R₃ are CH₃,) using the reaction conditions described above for Scheme 1.

The structures of various compounds of formula (1) (compounds 3.1 to 3.7) are set out below:

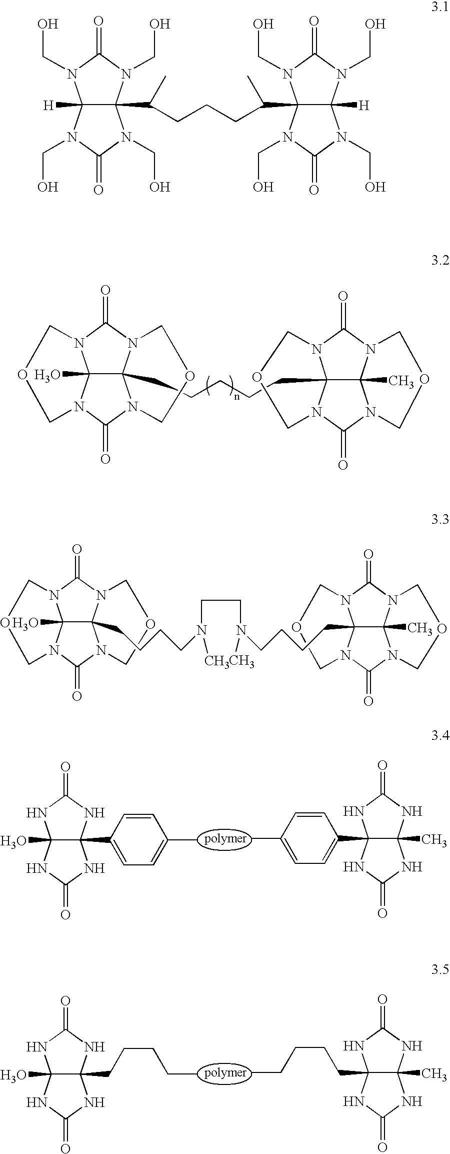

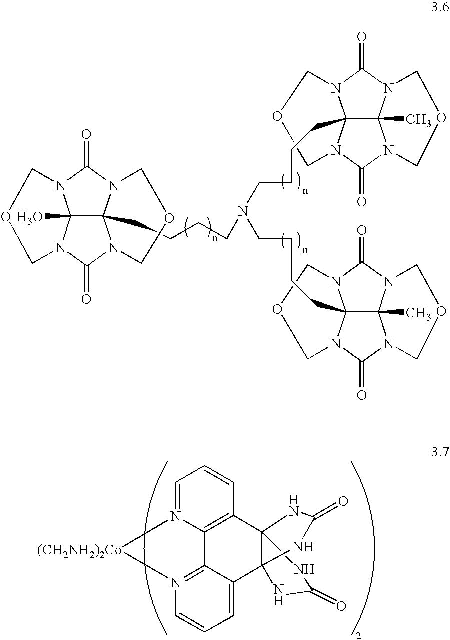

### EXAMPLE 14

A mixture of undecane-2,3,9,10-tetrone or a tetramethoxy acetal (1.4 gm) in acidified water pH 1 (0.5 mL)(optionally with a cosolvent THF(3 mL)) and urea (1.13 gm) was stirred at room temperature for several days. The solid linked glycoluril (compound 1.12) was collected by filtration and washed with methanol and dried (1 gm).

Preparation of compound 3.2. Aqueous 40% formaldehyde (0.7 mL) was added to the linked glycoluril (compound 1.12) (0.53 gm ) suspended in 8M HCl (1.2 mL) at ambient temperature. The stirred mixture was maintained at this temperature for 20 hr. Methanol (5 mL) was added to the homogeneous solution and the precipitate collected by filtration and dried in vacuo. The product (compound 3.2, where n=1) was used in the method of the present invention without further purification.

2. Formation of Compounds Comprising a Plurality of Cucurbituril Groups

In the following Examples 15 and 16, the “compound 3.2” was the compound 3.2 where n=1 prepared as described in Example 14.

### EXAMPLE 15

Compound 3.2 (350 mg) was added to a fine suspension of the unsubstituted glycoluril dimer (compound 2.2) (332 mg) in HCl 32% (5 mL). The mixture was stirred at room temperature for 2 h and a gel was formed. Heating the mixture to 95° C. for 3 hr gave a homogeneous solution. All volatile material was removed in vacuo to give a solid product. The solid product contained compounds comprising a plurality of cucurbituril groups.

An alternative procedure, which gave less crosslinking of the polymeric product, was carried out as described below.

Compound 3.2 (350 mg) was added to a fine suspension of the unsubstituted glycoluril dimer (compound 2.2) (663 mg) in HCl 32% (5 mL). The mixture was stirred at room temperature for 2 h to give a homogeneous mixture without forming a gel. Then (350 mg) of compound 3.2, was added and the mixture heated to 95° C. for 3 hr, after an initial period of 20 min at room temperature. All volatile material was removed in vacuo to give a solid product. This product was insoluble in water and salt solutions. The solid product contained compounds comprising a plurality of cucurbituril groups.

### EXAMPLE 16

Compound 3.2 (350 mg) was added to unsubstituted glycoluril (77 mg) in HCl 32% (3 mL). The mixture was stirred at room temperature for 2 h and a gel was formed. Heating the mixture to 95° C. for 3 hr gave a homogeneous solution. All volatile material was removed in vacuo to give a solid product. The solid product contained compounds comprising a plurality of cucurbituril groups.

### EXAMPLE 17

A compound of formula (1) having a structure similar to compound 3.5 was prepared by heating polyethylenimine (50% water solution 3 mL; the polyethylenimine having an average molecular weight of 2000 Daltons) with bisbromomethylglycoluril (compound 1.13, 100mg) for 12 hr. The resultant mixture was cooled in an ice bath, acidified with hydrochloric acid and the acid concentration increased to saturation by passing HCl gas into the mixture. At room temperature paraformaldehyde (37 mg) was added and the mixture maintained at room temperature for 12 hr. Then compound 2.2 (188 mg) and paraformaldehyde (18 mg) was added and the mixture heated to 90° C. for 3 hr. Evaporation of the solvent gave a solid product containing a plurality of predominately cucurbit[5]uril groups. The predominance of cucurbit[5]uril groups was demonstrated through gas adsorption experiments.

### EXAMPLE 18

A compound of formula (1) having a structure similar to compound 3.5 was prepared by heating polyethylenimine (50% water solution 3 mL; the polyethylemine having an average molecular weight of 2000 Daltons) with bisbromomethylglycoluril (compound 1.13, 100 mg) for 12 hr. The resultant mixture was cooled in an ice bath, acidified with hydrochloric acid and the acid concentration increased to saturation by passing HCl gas into the mixture. At room temperature paraformaldehyde (37 mg) was added and the mixture maintained at room temperature for 12 hr. Then compound 2.7 (240 mg) was added and the mixture heated to 90° C. for 3 hr. Evaporation of the solvent gave a solid product containing a plurality of predominately cucurbit[5]uril groups. The predominance of cucurbit[5]uril groups was demonstrated through gas adsorption uptake in particular acetylene/propane ratios.

Further examples of the method of the invention are provided by the following representative reaction schemes (1) to (6):

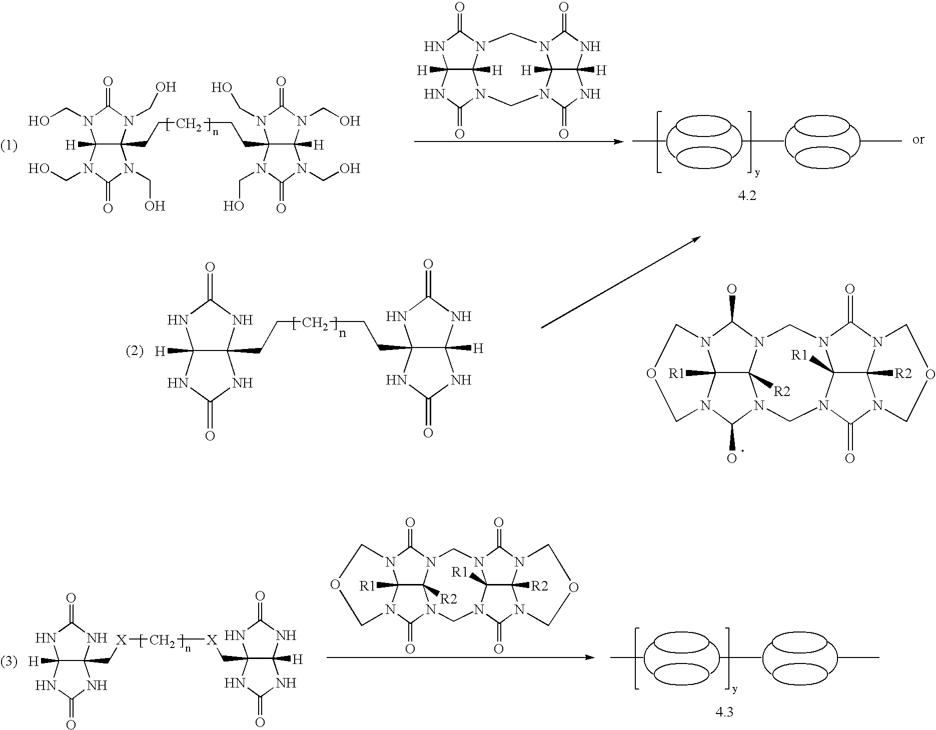

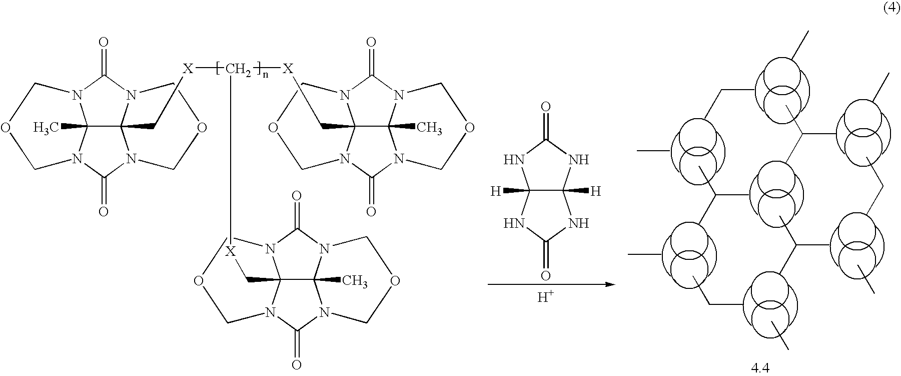

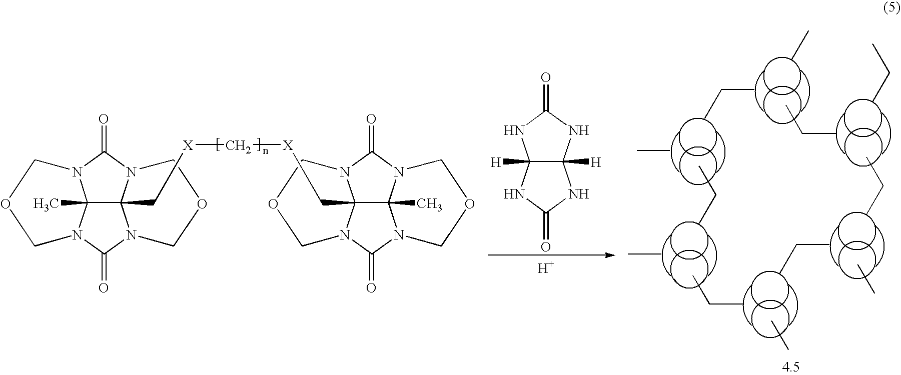

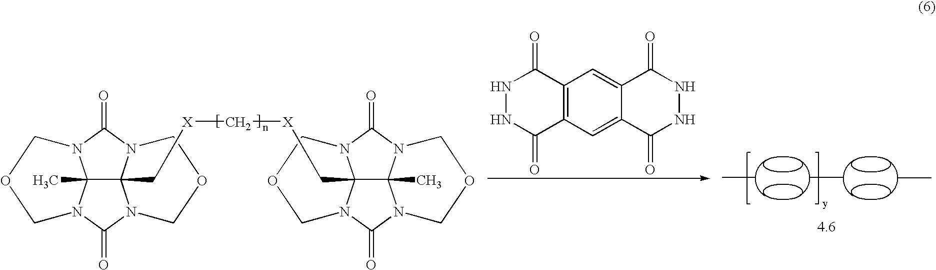

In representative reaction schemes (1) to (6), n and y are integers and X is a heteroatom such as N, S or O.

The representative reaction schemes (1) to (6) above are merely illustrative, and the products depicted are merely illustrative of the manner in which the cucurbituril groups may be linked in some of the compounds produced by the method of the invention.

The method of the present invention can be used to prepare compounds containing a plurality of cucurbituril groups. In some embodiments of the invention, the compound produced is a compound comprising a large number of linked cucurbituril groups. Whether the cucurbituril groups in the compounds prepared by the method of the present invention are linked in a linear, branched or cross-linked manner, and the extent of cross linking, depends on the groups A and the Additional Compounds (if any) used and the size of the cucurbituril groups formed. The distribution of sizes of the cucurbituril groups formed can be altered by the presence or absence of a templating compound. Compounds comprising a plurality of cucurbituril groups linked in a manner similar to that illustrated at 4.4 and 4.5 in illustrative reaction schemes (4) and (5) above predominantly occur when the cucurbituril groups formed are cucurbit[6]uril groups.

An advantage of the method of present invention is that the method involves the preparation of compounds containing a plurality of cucurbituril groups without requiring the initial production of cucurbiturils or cucurbituril analogues comprising a single cucurbituril group followed by the subsequent step of linking the cucurbiturils or cucurbituril analogues. This can result in cost and time savings.

The compounds comprising a plurality of cucurbituril groups prepared by the method of the present invention can be used for the same purposes as cucurbiturils as described in WO 00/68232. The compound comprising a plurality of cucurbituril groups prepared by the method of the present invention may, for example, be used to provide slow release of compounds complexed with the cucurbituril groups in the compound, for example, in formulations for the slow release of therapeutically active agents.

In some embodiments, the method of the present invention can be used to prepare compounds comprising a large number of linked cucurbituril groups. Such compounds are large molecules and are therefore typically less liable to being physically washed away by a liquid or gas passing past the compound than a smaller cucurbituril or cucurbituril analogue molecule comprising a single cucurbituril group. Further, in those applications where the compound comprising a plurality of cucurbituril groups is dissolved in a liquid, the high molecular weight of the compound means the compound can, if desired, be conveniently retained in a given environment in the liquid by use of an artificial or biological film or membrane.

In some embodiments, the compound comprising two or more cucurbituril groups produced by the method of the present invention may be shaped or otherwise formed into an article while maintaining the complexing property of the cucurbituril groups. For example, some compounds comprising a plurality of cucurbituril groups may be formed into films or beads. Such films can be used to partition solutions and gases and the cucurbituril groups on the film are able to selectively capture certain molecules or substances from the solution or gas, thus allowing selective substances to cross the film from one solution or gas to another

It will be appreciated by persons skilled in the art that numerous variations and/or modifications may be made to the invention as shown in the specific embodiments described herein without departing from the spirit or scope of the invention as broadly described. The specific embodiments described or exemplified herein are, therefore, to be considered in all respects as illustrative and not restrictive.

1.-15. (canceled)

16. A method for preparing a compound comprising a plurality of cucurbituril groups, the method comprising the steps of:
(a) forming a mixture comprising one or more compounds of the formula (1)
A-L-A (1)
wherein:
L is a linking group; and
each A is independently selected and is a group of the formula (A) 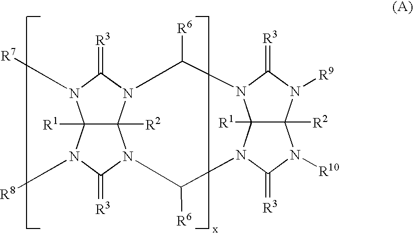
wherein:
for each unit of the formula (B) 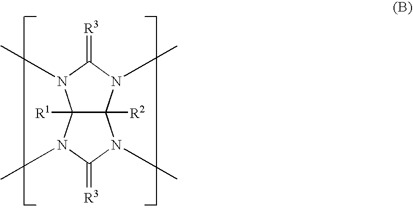
in formula (A),
R¹ and R² may be the same or different, and are each independently selected from a bond with L or
a univalent radical, or
R¹, R² and the carbon atoms to which they are bound together form an optionally substituted cyclic group, or
R¹ of one unit of the formula (B) and R² of an adjacent unit of the formula (B) together form a bond or a divalent radical,
and
each R³ is independently selected from the group consisting of ═O, ═S, ═NR′, ═CXZ, ═CZR′, ═CXR″ and ═CZ₂, wherein Z is an electron withdrawing group, X is halo, and R′ is selected from the group consisting of a bond with L, H, an optionally substituted straight chain, branched or cyclic, saturated or unsaturated hydrocarbon radical, or an optionally substituted heterocyclyl radical, and R″ is a bond with L;
each R⁶ is independently selected from the group consisting of a bond with L, H, alkyl and aryl; R⁷ and R⁸ may be the same or different and are independently selected from the group consisting of H and —CHR⁶OR⁶, or R⁷ and R⁸ together form the group —CHR⁶—O—CHR⁶—, where each R⁶ is independently selected from the group consisting of a bond with L, H, alkyl and aryl;
R⁹ and R¹⁰ may be the same or different and are independently selected from the group consisting of H and —CHR⁶OR⁶, or R⁹ and R¹⁰ together form the group —CHR⁶—O—CHR⁶—, where each R⁶ is independently selected from the group consisting of a bond with L, H, alkyl and aryl; and
x is 0 or an integer from 1 to 10;
provided that at least one R¹, R² or R⁶ is a bond with L or at least one R³ is ═NR″, ═CZR″ or ═CXR″ where R″ is a bond with L; and
an acid; and
(b) exposing the mixture to conditions effective for at least some of the groups A to react to form cucurbituril groups, thereby forming a compound comprising a plurality of cucurbituril groups.

17. A method according to claim 16, wherein step (b) comprises heating the mixture to a temperature from 20° C. to 120° C.

18. A method according to claim 16, wherein step (b) comprises contacting the one or more compounds of the formula (1) with a compound that can form bridges between groups A, and heating the mixture to a temperature from 20° C. to 120° C.

19. A method according to claim 18, wherein the compound that can form bridges between groups A is selected from the group consisting of compounds of the formula R⁵COR⁵ wherein each R⁵ is independently selected from the group consisting of H, alkyl and aryl, compounds of the formula R⁵OC(R⁵)₂OR⁵ wherein each R⁵ is independently selected from the group consisting of H, alkyl and aryl, trioxane, optionally substituted 3,4-dihydropyran and optionally substituted 2,3-dihydrofuran.

20. A method according to claim 18, wherein the compound that can form bridges between groups A is formaldehyde.

21. A method according to claim 16, wherein the mixture further comprises one or more compounds selected from compounds of the formula (6): 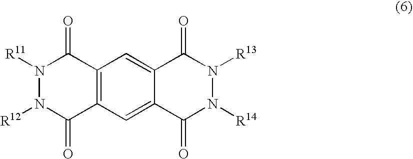
and compounds of the formula (2): 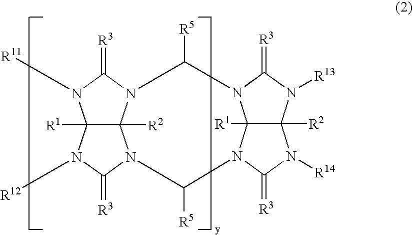
wherein:
for each unit of the formula (B): 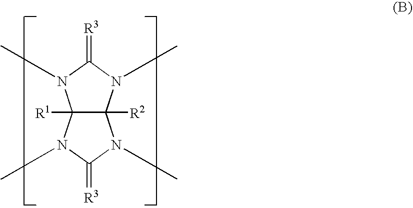
in the compound of formula (2),
R¹ and R² may be the same or different, and are each a univalent radical, or
R¹, R² and the carbon atoms to which they are bound together form an optionally substituted cyclic group, or
R¹ of one unit of the formula (B) and R² of an adjacent unit of the formula (B) together form a bond or a divalent radical,
and
each R³ is independently selected from the group consisting of ═O, ═S, ═NR, ═CXZ, ═CRZ or ═CZ₂, wherein Z is an electron withdrawing group, X is halo, and R is H, an optionally substituted straight chain, branched or cyclic, saturated or unsaturated hydrocarbon radical, or an optionally substituted heterocyclyl radical;
each R⁵ in formula (2) is independently selected from the group consisting of H, alkyl and aryl;
R¹¹ and R¹² may be the same or different and are independently selected from the group consisting of H and —CHR⁵OR⁵, or R¹¹ and R¹² together form the group —CHR⁵—O—CHR⁵—, where each R⁵ is independently selected and is as defined above,
R¹³ and R¹⁴ may be the same or different and are independently selected from the group consisting of H and —CHR⁵OR⁵, or R¹³ and R¹⁴ together form the group —CHR⁵—O—CHR⁵—, where each R⁵ is independently selected and is as defined as above; and
y is 0 or an integer from 1 to 9;
and wherein at least some of the cucurbituril groups formed are formed from a group A of one molecule of the formula (1), a group A of at least one other molecule of the formula (1) and one or more molecules of formula (2) or (6).

22. A method according to claim 21, wherein step (b) comprises heating the mixture to a temperature from 20° C. to 120° C.

23. A method according to claim 21, wherein step (b) comprises contacting the one or more compounds of the formula (1) with a compound that can form bridges between groups A, and between a group A and a compound of formula (2) or (6), and heating the mixture to a temperature from 20° C. to 120° C.

24. A method according to claim 23, wherein the compound that can form bridges between groups A, and between a group A and compound of formula (2) or (6), is selected from the group consisting of compounds of the formula R⁵COR⁵ wherein each R⁵ is independently selected from the group consisting of H, alkyl and aryl, compounds of the formula R⁵OC(R⁵)₂OR⁵ wherein each R⁵ is independently selected from the group consisting of H, alkyl and aryl, trioxane, optionally substituted 3,4-dihydropyran and optionally substituted 2,3-dihydrofuran.

25. A method according to claim 23 wherein the compound that can form bridges between groups A, and between a group A and compound of formula (2) or (6), is formaldehyde.

26. A method according to claim 16, wherein R³ is O and R⁶ is H.

27. A method according to claim 16 wherein L is a polymer.

28. A method according to claim 16 wherein L is a group of the formula —(CR₂)ₐ-(E-(CR₂)_{b}—)_{c}(CR₂)_{d}— or —(CR₂)ₐ-(E-(CR═CR)_{b}—)_{c}(CR₂)_{d}—
wherein:
E is —O—, —NR—, —S—, a saturated or unsaturated divalent hydrocarbon radical, or an optionally substituted aliphatic or aromatic divalent heterocyclyl radical;
R is H, an optionally substituted straight chain, branched or cyclic, saturated or unsaturated hydrocarbon radical or an optionally substituted heterocyclyl radical; and
a, b, c and d are each 0 or an integer from 1 to 30;
provided that not all of a, b, c and d are 0.

29. A method according to claim 16 wherein L is —(CH₂)ₙ—, —(CH═CH)ₙ—, —O—, —NH—,
—CH₂—NH—, —CH(CH₃)(CH₂)ₙCH(CH₃)— or
—(CH₂)ₙ—N(CH₃)CH₂CH₂N(CH₃)—(CH₂)ₚ—,
where n and p are an integer.

30. A compound comprising a plurality of cucurbituril groups produced by the method of claim 16.
